(19) United States
(12) Patent Application Publication
(10) Pub. No.: US 2008/0139612 A1
(43) Pub. Date: Jun. 12, 2008

(54) Chemical Compound

(75) Inventors: Alan Wellington Faull, Macclesfield (GB); Steven Swallow, Macclesfield (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/953,197

(22) Filed: Dec. 10, 2007

Related U.S. Application Data
(60) Provisional application No. 60/869,436, filed on Dec. 11, 2006.

(51) Int. Cl. A61K 31/4545; A61K 31/454; C07D 401/14; A61P 11/06; A61P 37/08; A61P 19/02; A61P 9/10

(52) U.S. Cl. 514/316; 546/187

(57) ABSTRACT

The present invention relates to 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine (I):

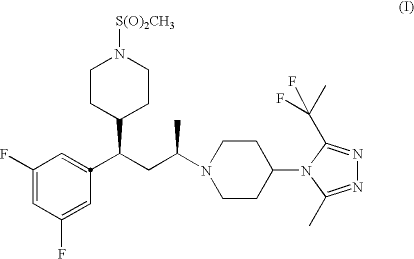

or a pharmaceutically acceptable salt thereof, as well as processes for the preparation of this compound and its use in the treatment of CCR5 disease states.

This application claims the benefit under 35 U.S.C. § 119(e) of U.S. Provisional Application No. 60/869,436, filed on Dec. 11, 2006, which is incorporated herein by reference in its entirety.

The present invention relates to a piperidine compound having pharmaceutical activity, to processes for preparing such a compound, to pharmaceutical compositions comprising such a compound and to the use of such a compound as an active therapeutic agent.

Chemokines are chemotactic cytokines that are released by a wide variety of cells to attract macrophages, T cells, eosinophils, basophils and neutrophils to sites of inflammation and also play a role in the maturation of cells of the immune system. Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1α and MIP-1β and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

Pharmaceutically active piperidine derivatives are disclosed in PCT/SE2005/000574 (WO 2005/101989). One of the disclosed compounds is 1-{(3R)-3-(3,5-difluorophenyl)-3-[1-(methylsulfonyl)piperidin-4-yl]propyl}-4-[3-methyl-5-(trifluoromethyl)-4H-1,2,4-triazol-4-yl]piperidine (Comparator Compound A). The compound of the present invention has particularly advantageous potency and/or other beneficial pharmaceutical properties over Comparator Compound A.

The present invention provides 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine (I):

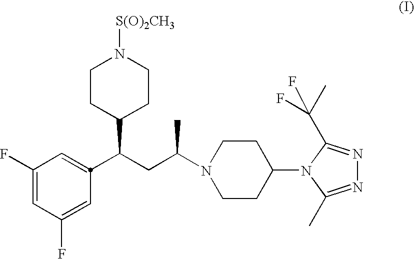

or a pharmaceutically acceptable salt thereof.

Suitable pharmaceutically acceptable salts include acid addition salts (adducts) such as a hydrochloride, hydrobromide, phosphate, acetate, fumarate, succinate, maleate, tartrate, citrate, oxalate, methanesulphonate, p-toluenesulphonate or formate.

The compound of the invention may exist as a solvate (such as a hydrate) and the present invention encompasses all such solvates.

The compound of the present invention can be prepared by any of the suitable processes disclosed in PCT/SE2005/000574 (WO 2005/101989).

For example, the compound of the present invention can be prepared by reaction of a compound of formula (II):

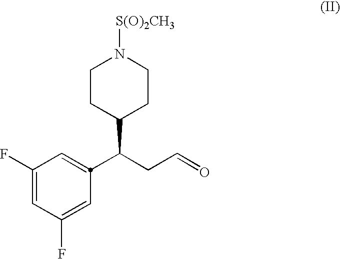

with a compound of formula (III) in the presence of an appropriate triazole (for example 1,2,3-triazole or benzotriazole):

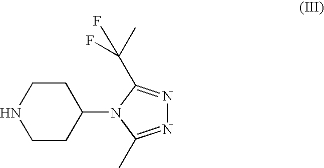

followed by reaction with an appropriate organometallic reagent (for example methyl magnesium bromide).

A compound of formula (III) can be prepared by removal of the protecting group (PG) from a compound of formula (IV):

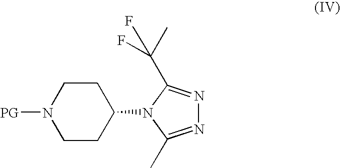

for example where PG is benzyloxylcarbonyl or benzyl removal may be effected by hydrogenation (for example hydrogen in the presence of palladium on carbon catalyst); where PG is tert-butyloxycarbonyl removal may be effected by treatment with acid (such as hydrochloric acid or trifluoroacetic acid).

A compound of formula (IV): can be prepared from a compound of formula (V):

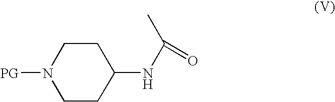

using a “one-pot”, two-step procedure by first activating the amide with, for example, phosphorous pentachloride, and reacting the product so formed with an acyl hydrazide, then by cyclising in the presence of an acid at elevated temperature (such as acetic acid in refluxing toluene).

The compound of the invention can be prepared by alkylation of a compound of formula (VI):

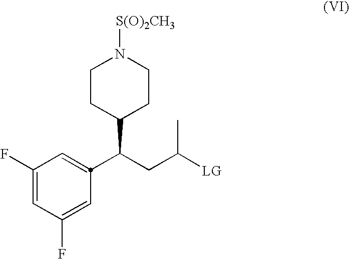

wherein LG is a leaving group; with a compound of formula (III) in the presence of a suitable base (such as potassium carbonate or triethylamine) in a suitable solvent (such as acetonitrile or THF) at room temperature (for example 10-30° C.).

The compound of the invention can be prepared by reductive amination of a compound of formula (VII):

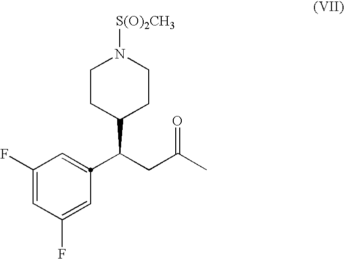

with a compound of formula (III), in the presence of a reducing reagent (such as NaBH(OAc)₃, wherein Ac is C(O)CH₃) and an appropriate Lewis acid (such as Ti(OPr)₄ in a suitable solvent (EtOH).

The starting materials for these preparative methods are either commercially available or can be prepared by literature methods, adapting literature methods, or adapting Methods herein described.

The compound of the present invention has activity as a pharmaceutical, in particular as a modulator (such as agonist, partial agonist, inverse agonist or antagonist) of chemokine receptor (for example CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

The compound of the present invention is also of value in inhibiting the entry of viruses (such as human immunodeficiency virus (HIV)) into target cells and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, for use in a method of treatment of a warm blooded animal (such as man) by therapy (including prophylaxis).

According to a further feature of the present invention there is provided a method for modulating chemokine receptor activity (for example CCR5 receptor activity) in a warm blooded animal, such as man, in need of such treatment, which comprises administering to said animal an effective amount of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof.

The present invention also provides the use of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, as a medicament, for example a medicament for the treatment of transplant rejection, respiratory disease, psoriasis or rheumatoid arthritis (such as rheumatoid arthritis). [Respiratory disease is, for example, COPD, asthma {such as bronchial, allergic, intrinsic, extrinsic or dust asthma, particularly chronic or inveterate asthma (for example late asthma or airways hyper-responsiveness)} or rhinitis {acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}; and is particularly asthma or rhinitis].

In another aspect the present invention provides the use of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, in the manufacture of a medicament for use in therapy (for example modulating chemokine receptor activity (such as CCR5 receptor activity (for example rheumatoid arthritis)) in a warm blooded animal, such as man).

The invention also provides 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, for use as a medicament, for example a medicament for the treatment of rheumatoid arthritis.

In another aspect the present invention provides the use of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, in the manufacture of a medicament for use in therapy (for example modulating chemokine receptor activity (such as CCR5 receptor activity (for example rheumatoid arthritis)) in a warm blooded animal, such as man).

The invention further provides the use of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthritides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (pain and connective tissue remodelling of musculoskeletal disorders due to injury [for example sports injury] or disease) arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
(4) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia greata or vernal conjunctivitis;
(5) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(6) (Allograft rejection) acute and chronic following, for example, transplantation of kidney, heart, liver, lung, bone marrow, skin or cornea; or chronic graft versus host disease; and/or
(7) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle;
   in a warm blooded animal, such as man.

The present invention further provides a method of treating a chemokine mediated disease state (for example a CCR5 mediated disease state) in a warm blooded animal, such as man, which comprises administering to a mammal in need of such treatment an effective amount of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof.

In order to use 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof (active ingredient), and a pharmaceutically acceptable adjuvant, diluent or carrier. In a further aspect the present invention provides a process for the preparation of said composition which comprises mixing 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine with a pharmaceutically acceptable adjuvant, diluent or carrier. Depending on the mode of administration, the pharmaceutical composition will comprise, for example, from 0.05 to 99% w (percent by weight), such as from 0.05 to 80% w, for example from 0.10 to 70% w (such as from 0.10 to 50% w), of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, all percentages by weight being based on total composition.

The pharmaceutical composition of this invention may be administered in standard manner for the disease condition that it is desired to treat, for example by topical (such as to the lung and/or airways or to the skin), oral, rectal or parenteral administration. For these purposes the compound of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine.

In another aspect a pharmaceutical composition of the invention is one suitable for intravenous, intraarticular, subcutaneous or intramuscular injection.

Each patient may receive, for example, an intravenous, intraarticular, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intravenous, intraarticular, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof or a solvent thereof (hereafter Compound X), for therapeutic or prophylactic use in humans:

(a)

| Tablet I | mg/tablet |
|---|---|
| Compound X | 100 |
| Lactose Ph.Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph.Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph.Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph.Eur. | 389 |
| Croscarmellose sodium | 100 |
| Magnesium stearate | 1.0 |

(e)

| Injection I | (50 mg/mL) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or EtOH or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention further relates to combination therapies or compositions wherein 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, or a pharmaceutical composition comprising 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, is administered concurrently (possibly in the same composition) or sequentially with an agent for the treatment of any one of the above disease states.

In particular, for the treatment of the inflammatory diseases rheumatoid arthritis, psoriasis, inflammatory bowel disease, COPD, asthma and allergic rhinitis the compound of the invention can be combined with a TNF-α inhibitor (such as an anti-TNF monoclonal antibody (such as Remicade, CDP-870 and D.sub2.E.sub7.), or a TNF receptor immunoglobulin molecule (such as Enbrel.reg.)), a non-selective COX-1/COX-2 inhibitor (such as piroxicam or diclofenac; a propionic acid such as naproxen, flubiprofen, fenoprofen, ketoprofen or ibuprofen; a fenamate such as mefenamic acid, indomethacin, sulindac or apazone; a pyrazolone such as phenylbutazone; or a salicylate such as aspirin), a COX-2 inhibitor (such as meloxicam, celecoxib, rofecoxib, valdecoxib or etoricoxib) low dose methotrexate, lefunomide; ciclesonide; hydroxychloroquine, d-penicillamine or auranofin, or parenteral or oral gold.

The present invention still further relates to the combination of the 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof, together with:
a leukotriene biosynthesis inhibitor, a 5-lipoxygenase (5-LO) inhibitor or a 5-lipoxygenase activating protein (FLAP) antagonist, such as zileuton, ABT-761, fenleuton, tepoxalin, Abbott-79175, Abbott-85761, an N-(5-substituted)-thiophene-2-alkylsulfonamide, a 2,6-di-tert-butylphenol hydrazones, a methoxytetrahydropyran such as Zeneca ZD-2138, SB-210661, a pyridinyl-substituted 2-cyanonaphthalene compound such as L-739,010; a 2-cyanoquinoline compound such as L-746,530; an indole or quinoline compound such as MK-591, MK-886 or BAY x 1005;
a receptor antagonist for a leukotriene LTB.sub4., LTC.sub4., LTD.sub4. or LTE.sub4. selected from the group consisting of a phenothiazin-3-one such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A) or BAY x 7195;
a PDE4 inhibitor including an inhibitor of the isoform PDE4D;
an antihistaminic H.sub1. receptor antagonist such as cetirizine, loratadine, desloratadine, fexofenadine, astemizole, azelastine or chlorpheniramine;
a gastroprotective H.sub2. receptor antagonist;
an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride or ethylnorepinephrine hydrochloride;
an anticholinergic agent such as ipratropium bromide, tiotropium bromide, oxitropium bromide, pirenzepine or telenzepine;
a β.sub1.- to β.sub4.-adrenoceptor agonist such as metaproterenol, isoproterenol, isoprenaline, albuterol, salbutamol, formoterol, salmeterol, terbutaline, orciprenaline, bitolterol mesylate or pirbuterol, or a methylxanthanine including theophylline and aminophylline; sodium cromoglycate; or a muscarinic receptor (M1, M2, and M3) antagonist;
an insulin-like growth factor type I (IGF-1) mimetic;
an inhaled glucocorticoid with reduced systemic side effects, such as prednisone, prednisolone, flunisolide, triamcinolone acetonide, beclomethasone dipropionate, budesonide, fluticasone propionate or mometasone furoate;
an inhibitor of a matrix metal loprotease (MMP), such as a stromelys in, a collagenase, or a gelatinase or aggrecanase; such as collagenase-1 MMP-1), collagenase-2 (MMP-8), collagenase-3 (MMP-13), stromelysin-1 (MMP-3), stromelysin-2 (MMP-10), and stromelysin-3 (MMP-11) or MMP-12;
a modulator of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family;
an osteoporosis agent such as roloxifene, droloxifene, lasofoxifene or fosomax;
an immunosuppressant agent such as FK-506, rapamycin, cyclosporine, azathioprine or methotrexate;
a compound useful in the treatment of AIDS and/or HIV infection for example: an agent which prevents or inhibits the viral protein gp120 from engaging host cell CD4 {such as soluble CD4 (recombinant); an anti-CD4 antibody (or modified/recombinant antibody) for example PRO542; an anti-group 120 antibody (or modified/recombinant antibody); or another agent which interferes with the binding of group 120 to CD4 for example BMS806}; an agent which prevents binding to a chemokine receptor, other than CCR5, used by the HIV virus {such as a CXCR4 agonist or antagonist or an anti-CXCR4 antibody}; a compound which interferes in the fusion between the HIV viral envelope and a cell membrane {such as an anti-group 41 antibody; enfuvirtide (T-20) or T-1249}; an inhibitor of DC-SIGN (also known as CD209) {such as an anti-DC-SIGN antibody or an inhibitor of DC-SIGN binding}; a nucleoside/nucleotide analogue reverse transciptase inhibitor {for example zidovudine (AZT), nevirapine, didanosine (ddI), zalcitabine (ddC), stavudine (d4T), lamivudine (3TC), abacavir, adefovir or tenofovir (for example as free base or as disoproxil fumarate)}; a non-nucleoside reverse transciptase inhibitor {for example nevirapine, delavirdine or efavirenz}; a protease inhibitor {for example ritonavir, indinavir, saquinavir (for example as free base or as mesylate salt), nelfinavir (for example as free base or as mesylate salt), amprenavir, lopinavir or atazanavir (for example as free base or as sulphate salt)}; a ribonucleotide reductase inhinbitor {for example hydroxyurea}; or an antiretroviral {for example emtricitabine}; or,
an existing therapeutic agent for the treatment of osteoarthritis, for example a non-steroidal anti-inflammatory agent (hereinafter NSAID's) such as piroxicam or diclofenac, a propionic acid such as naproxen, flubiprofen, fenoprofen, ketoprofen or ibuprofen, a fenamate such as mefenamic acid, indomethacin, sulindac or apazone, a pyrazolone such as phenylbutazone, a salicylate such as aspirin, a COX-2 inhibitor such as celecoxib, valdecoxib, rofecoxib or etoricoxib, an analgesic or intra-articular therapy such as a corticosteroid or a hyaluronic acid such as hyalgan or synvisc, or a P2X7 receptor antagonist.

The present invention still further relates to the combination of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine, or a pharmaceutically acceptable salt thereof together with: (i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitor including a VLA-4 antagonist; (vi) a cathepsin; (vii) a MAP kinase inhibitor; (viii) a glucose-6 phosphate dehydrogenase inhibitor; (ix) a kinin-B.sub1.- and B.sub2.-receptor antagonist; (x) an anti-gout agent, e.g., colchicine; (xi) a xanthine oxidase inhibitor, e.g., allopurinol; (xii) an uricosuric agent, e.g., probenecid, sulfinpyrazone or benzbromarone; (xiii) a growth hormone secretagogue; (xiv) a transforming growth factor (TGFβ); (xv) a platelet-derived growth factor (PDGF); (xvi) a fibroblast growth factor, e.g., basic fibroblast growth factor (bFGF); (xvii) a granulocyte macrophage colony stimulating factor (GM-CSF); (xviii) a capsaicin cream; (xix) a Tachykinin NK.sub1. and NK.sub3. receptor antagonist selected from the group consisting of NKP-608C; SB-233412 (talnetant); and D-4418; (xx) an elastase inhibitors selected from the group consisting of UT-77 and ZD-0892; (xxi) a TNFα converting enzyme inhibitor (TACE); (xxii) an induced nitric oxide synthase inhibitor (iNOS); or (xxiii) a chemoattractant receptor-homologous molecule expressed on TH2 cells (a CRTH2 antagonist).

The invention will now be illustrated by the following non-limiting Examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 mL disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.
(iv) in general, the course of reactions was followed by TLC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at 400 MHz using perdeuterio DMSO (CD₃SOCD₃) as the solvent unless otherwise stated; coupling constants (J) are given in Hz;
(vii) chemical symbols have their usual meanings; SI units and symbols are used;
(viii) solvent ratios are given in percentage by volume;
(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion-(M+H)⁺;
(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion-(M+H)⁺;
(xi) the compounds of the Examples and Methods were named using the IUPAC name program from Advanced Chemistry Development Inc, version 6.00; and,
(xii) the following abbreviations are used:

THF Tetrahydrofuran;

DCM Dichloromethane

DIPE Di-iso-propyl ether

DIBAL Di-iso-butylaluminium hydride

DMSO Dimethylsulfoxide

IPA Iso-propanol

R-BINAP (R)-2,2′-Bis(diphenylphosphino)-1,1′-binaphthyl TPAP Tetrapropylammonium perruthenate

Mol eq Molar equivalents

Rel vol Relative volume

MTBE Methyl tert-butylether

### EXAMPLE 1

### 4-[3-(1,-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine

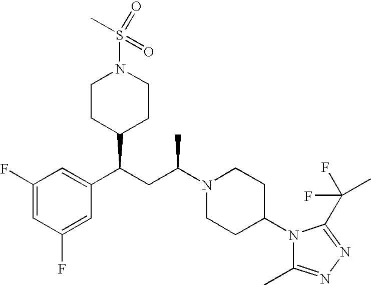

### Method A

4-[3-(1,1-difluoroethyl)-5-methyl-1,2,4-triazol-4-yl]piperidine (21.0 g, 1.1 eq), (3R)-3-(3,5-difluorophenyl)-3-(1-methylsulfonyl-4-piperidyl)propanal (27.5 g, 1.0 eq) and 1,2,3-Triazole (5.8 ml, 1.2 eq) were heated at reflux (110° C.) in toluene (412 ml, 10 vol) under Dean-Stark conditions for 3.5 hours, (˜1.5 ml of water collected). The reaction was cooled to 0° C. and methyl magnesium bromide (3M in diethylether) (110.5 ml, 4.0 eq) was then added over 20 minutes, not allowing the internal temperature to exceed 20° C. (exothermic 0° C.-8° C.). The reaction was allowed to stir at 20° C. for 1 hour. The reaction was cooled back to 0° C. then carefully quenched with aqueous saturated ammonium chloride solution (250 ml, 5 vol) (very exothermic 0° C.-30° C., after a few drops, and lots of gas effervescence). Allowed to cool back to 20° C. then ethyl acetate (500 ml, 10 vol) was added. The organic layer was separated and the aqueous layer extracted with ethyl acetate (1.0 litres, 20 vol). The organics were combined, washed with water (1.0 litres, 20 vol), 50% brine/water (1.0 litres, 20 vol), dried (magnesium sulphate), filtered and the solvent removed in vacuo gave 50.2 g. The diastereoisomers were separated by column chromatography on the Companion XL (1500 g silica column) eluting with 5%-20% methanol/ethyl acetate gradient

The first eluted diastereomer (isomer A) gave 19.5 g of white solid and the second eluted diastereomer (isomer B, title compound) gave 21.5 g of white solid.

Isomer A: 4-[3-(1,1-difluoroethyl)-5-methyl-4H-1,2,4-triazol-4-yl]-1-{(1S,3R)-3-(3,5-difluorophenyl)-1-methyl-3-[1-(methylsulfonyl)piperidin-4-yl]propyl}piperidine; ¹H NMR (400 MHz, DMSO) δ 0.78 (d, 3H), 0.97-1.37 (m, 3H), 1.38-1.68 (m, 2H), 1.70-2.31 (m, 12H), 2.31-2.75 (m, 7H), 2.75-2.91 (m, 4H), 3.39-3.52 (m, 1H), 3.52-3.67 (m, 1H), 4.14-4.32 (m, 1H), 6.87-7.12 (m, 3H)

Isomer B: (title compound), 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine; ¹H NMR (400 MHz, DMSO) δ 0.92 (d, 3H), 0.97-1.25 (m, 2H), 1.25-1.37 (m, 1H), 1.48-1.92 (m, 6H), 1.91-2.02 (m, 2H), 2.05-2.34 (m, 6H), 2.44-2.68 (m, 7H), 2.69-2.88 (m, 4H), 3.46 (d, 1H), 3.57 (d, 1H), 4.13-4.25 (m, 1H), 6.84-7.14 (m, 3H)

### Method B

In a similar manner to Method A, but using benzotriazole instead of 1,2,3-Triazole, Isomer A (yield 11%) and Isomer B (yield 12%) were prepared.

The (3R)-3-(3,5-difluorophenyl)-3-(1-methylsulfonyl-4-piperidyl)propanal used as starting material was prepared as follows:

### Step 1.

### Preparation of 1-methanesulfonyl-4-(ethoxycarbonyl)-piperidine

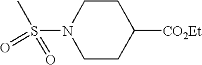

Ethyl isonipecotate (1 mol eq) was charged to a reaction vessel followed by a line wash of DCM (1 rel vol). Triethylamine (1 mol eq) was charged to the vessel followed by a line wash of DCM (1 rel vol). DCM (5 rel vol) was charged to the vessel and the reaction mixture cooled to between 0 and 5° C. A solution of methane sulfonyl chloride (1 mol eq) in DCM (2 rel vol) followed by a line wash of DCM (1 rel vol) was added to the vessel maintaining the temperature between 1 and 10° C. The reaction mixture was stirred at between 0 and 10° C. until the reaction was complete. Purified water (5 rel vol) was charged to the reaction mixture and stirred for 15 minutes at between 5 and 10° C. The resulting phases were separated and the organic phase was concentrated to approximately 4.5 rel vol by atmospheric distillation. The concentrate was clarified, and then DIPE (10 rel vol) was added and the reaction concentrated again to approximately 4.5 rel vols by reduced pressure distillation. Another portion of DIPE (10 rel vol) was added and the resulting suspension was stirred at ambient temperature for at least 60 minutes. The solid was isolated by filtration, washed with DIPE (2 rel vols) and then dried at ambient temperature to give the sub-titled compound in approximately 93% yield.

¹H NMR (400 MHz, DMSO-d⁶) δ 4.05 (q, J=7.1 Hz, 2H), 3.46 (d, J=12.0 Hz, 2H), 2.81 (s, 3H), 2.76 (t, J=11.5 Hz, 2H), 2.48-2.38 (m, 1H), 1.90 (d, J=13.3 Hz, 2H), 1.56 (dd, J=35.4, 3.5 Hz, 2H), 1.16 (t, J=7.2 Hz, 3H).

### Step 2.

### Preparation of (1-methanesulfonylpiperidin-4-yl)methanol

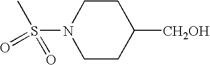

1-Methanesulfonyl-4-(ethoxycarbonyl)-piperidine (1 mol eq) was charged to a reaction vessel followed by a line wash of THF (6 rel vols). The reaction mixture was cooled to between 0 and 10° C. A solution of lithium aluminium hydride (1M in THF, 0.75 mol eq) followed by a line wash of THF (1 rel vol) was added to the vessel, keeping the temperature between 0° C. and 20° C., and then the reaction mixture was warmed to ambient temperature and stirred until the reaction was complete. The reaction mixture was cooled to between 0 and 2° C. Purified water (1 rel vol) was then charged to the vessel maintaining the temperature between 0° to 10° C. The pH of the reaction was adjusted to <2 by charging 5M HCl, maintaining the temperature between 0 and 10° C. The reaction mixture was warmed to room temperature, stirred for at least 15 minutes and then the phases separated. DCM (5 rel vol) was charged to the aqueous phase, stirred for at least 15 minutes and the phases separated. The first organic (THF) phase was concentrated to approximately 3.5 rel vols by vacuum distillation at 40° C. The second organic (DCM) phase was added to the concentrate, the phases separated and the organic phase concentrated to approximately 3.5 rel vol by atmospheric distillation. DIPE (10 rel vol) was added to the residue from the distillation at 40 to 45° C. After concentration to approximately 5 rel vol by vacuum distillation more DIPE (5 rel vol) was added and the resulting slurry cooled to ambient temperature and stirred for approximately 60 minutes. The sub-titled compound was isolated by filtration, washed with DIPE (2×1 rel vol) and dried at ambient temperature to give the sub-titled compound in approximately 87% yield.

¹H NMR (400 MHz, CDCl₃) δ 3.84 (dd, J=9.6, 2.2 Hz, 2H), 3.54 (d, J=4.9 Hz, 2H), 2.78 (s, 3H), 2.67 (t, J=12.0 Hz, 2H), 1.70-1.56 (m, 2H), 1.54 (s, 1H), 1.36 (qd, J=12.5, 4.2 Hz, 2H).

### Step 3.

### Preparation of (1-methanesulfonylpiperidin-4-yl)methanol

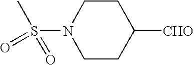

### Method A

(1-Methanesulfonylpiperidin-4-yl)methanol (1 mol eq) was dissolved in DCM (5 rel vol) in a reaction vessel followed by a line wash of DCM (1.2 rel vol). Pyridinium chlorochromate (1 mol eq) as a slurry in DCM (10 rel vol) was added followed by DCM (5×1.2 rel vol) as line washes. The reaction mixture was stirred overnight at ambient temperature, after which water (18.3 rel vol) was added and the phases separated and the DCM phase passed through a short “pad” of silica eluting with EtOAc. The solvent was evaporated from the filtrate to leave the sub-titled compound as a solid in approximately 40% yield.

### Method B

(1-Methanesulfonylpiperidin-4-yl)methanol (1 mol eq) and molecular sieves (2.5 weight eq) and TPAP (0.05 mol eq) were charged to a reaction vessel with DCM (30 rel vols). N-Methyl-morpholine N-oxide (1.5 mol eq) was dissolved in DCM (5 rel vols) in a separate vessel and added to the first vessel, keeping the temperature below 24° C. Once the reaction had reached completion the reaction mixture was filtered through celite and the solvent evaporated from the filtrate in vacuo to leave the sub-titled as a white solid in approximately 40% yield.

### Method C

1-Methanesulfonyl-4-(ethoxycarbonyl)-piperidine (1 mol eq) was weighed into a reaction vessel with DCM (16 rel vol) and cooled to −77° C. DIBAL (1M in THF, 1.5 mol eq) was added slowly, keeping the temperature of the reaction below −75° C. After 3 hours another charge of DIBAL solution (1.5 mol eq) was added at low temperature. Once the reaction had reached completion the reaction mixture was quenched with ammonium chloride solution (20% w/w, 2 rel vol), keeping the temperature below −67° C. After stirring at this temperature for 30 minutes, HCl (2M, 2 rel vol) was added, again keeping the temperature below −68° C. The resulting mixture was allowed to warm to ambient temperature overnight to give a white slurry. Water, HCl (5M) and brine were added until the precipitate dissolved. The layers were separated and solvent was removed from the organic layer by evaporation in vacuo to give the sub-titled compound in approximately 65% yield (contaminated with 1-methanesulfonylpiperidin-4-yl)methanol).

### Method D

A solution of DCM (5 rel vol) and oxalyl chloride (3 mol eq) was cooled to below −70° C. In a separate vessel, DCM (2 rel vol) and DMSO (6 mol eq) were mixed before addition to the oxalyl chloride solution via a syringe, keeping the temperature below −64° C. during the addition. After stirring for 10 minutes a solution of (1-methanesulfonylpiperidin-4-yl)methanol (1 mol eq) in DCM (5 rel vol) and DMSO (0.5 rel vol) was added, keeping the temperature below −60° C. during the addition. The reaction mixture was held at −70° C. for 40 minutes before adding triethylamine (7.5 mol eq) slowly via a syringe. The reaction mixture was allowed to warm to room temperature overnight. HCl (2M, 5 rel vol) was added while cooling the reaction in an ice-water bath. DCM (5 rel vol) was added before separating the layers and washing the DCM layer with: HCl (2M, 5 rel vol); then sodium bicarbonate solution (saturated, 5 rel vol); and finally brine (5 rel vol). The organic solvent was removed from the organic phase in vacuo to leave the sub-titled in approximately 75% yield.

¹H NMR (400 MHz, CDCl₃) δ 9.69 (s, 1H), 3.68-3.54 (m, 2H), 2.96 (ddd, J=12.3, 9.7, 2.8 Hz, 2H), 2.78 (s, 3H), 2.43 (dquintet, J=9.5, 4.7 Hz, 1H), 2.10-2.00 (m, 2H), 1.81 (dtd, J=13.8, 9.8, 3.9 Hz, 2H).

### Step 4.

### Preparation of iso-propyl 3-(1-methanesulfonylpiperidin-4-yl)propenoate

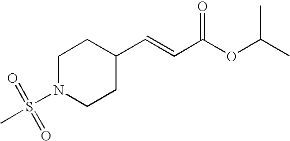

(1-Methanesulfonylpiperidin-4-yl)methanal (1 mol eq) was charged to a reaction vessel followed by a line wash of toluene (11 rel vol). Piperidine (0.1 mol eq) was charged to the vessel followed by a line wash of toluene (0.5 rel vol), and the reaction mixture heated to between 85 and 95° C. A solution of the iso-propyl malonic acid (1.25 mol eq) in toluene (prepared as described above) was added in 10 approximately equal portions over 6 to 8 hours and the reaction mixture was stirred at to between 85 and 95° C. until it reached completion. The reaction mixture was then cooled to between 40 and 50° C. and HCl (0.5M, 3 rel vol) was added to the reaction maintaining the temperature between 40 and 50° C. After stirring for at least 15 minutes the phases were separated. Sodium bicarbonate (0.5M, 3 rel vol) was added to the organic phase, still maintaining the temperature between 40 and 50° C. The 2-phase mixture was stirred for at least 15 minutes before separating the phases and washing the organic phase with water (3 rel vol). The organic phase was then concentrated to approximately 16 rel vols by vacuum distillation at between 40 and 50° C. Toluene (3.5 rel vol) was charged, the solution clarified at between 40 and 50° C. and then concentrated to approximately 7 rel vol by vacuum distillation. The mixture was then cooled to between 0 and 10° C. and stirred for at least 60 minutes at this temperature before isolating the sub-titled compound by filtration and washing the residue with toluene (2 rel vol) at between 0 and 10° C. The solid was dried to leave the sub-titled compound in approximately 59% yield.

¹H NMR (400 MHz, CDCl₃) δ 6.87 (dd, J=15.8, 6.5 Hz, 1H), 5.81 (dd, J=15.8, 0.9 Hz, 1H), 5.07 (quintet, J=6.2 Hz, 1H), 3.82 (d, J=12.0 Hz, 2H), 2.79 (s, 3H), 2.74 (td, J=12.0, 2.4 Hz, 2H), 2.36-2.17 (m, 1H), 1.95-1.80 (m, 2H), 1.57 (ddd, J=24.9, 11.7, 4.0 Hz, 2H), 1.27 (d, J=6.4 Hz, 6H).

### Step 5.

### Preparation of iso-propyl (3R)-3-(3,5-difluorophenyl)-3-[1-(methylsulfonyl)piperidin-4-yl]propanoate

Method A: (using 3,5-difluorophenylboronic Acid)

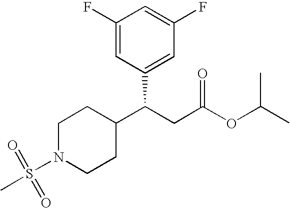

A catalyst solution was prepared by charging R-BINAP (0.045 mol eq) and bis(1,5-cyclooctadienerhodium chloride), (0.02 mol eq) to a vessel followed by THF (2.8 rel. vols). The mixture was stirred to achieve full dissolution.

To a larger reaction vessel was charged iso-propyl 3-(1-methanesulphonylpiperidin-4-yl)propenoate (1 mol eq), 3,5-difluorophenylboronic acid (1.35 mol eq) and potassium carbonate (1.35 mol eq). THF (7.8 rel vols) and IPA (1 mol eq) were then charged and the mixture was heated to 60° C. The catalyst solution was then added to this mixture, and a line wash of THF (1.4 rel vols) was used to facilitate this transfer. The resulting mixture was then held at 60° C. for 2 hours. The reaction mixture was cooled to room temperature a solution of L-cysteine (0.9 rel wt) in water (12 rel vols), was added. The resulting mixture was stirred at room temperature overnight. The phases were then separated and the organic portion was concentrated to a volume of 3.5 rel vols. IPA (10.5 rel vols) was then charged and the batch was then concentrated again to a volume of 3.5 rel vols. Further IPA (10.5 rel vols) was charged, and again the batch was concentrated to a volume of 3.5 rel vols. Finally a further 10.5 rel vols of IPA was charged, and the resulting mixture was held at 30-35° C. for 15-30 minutes, then heated to 70° C. The mixture was then filtered into a crystallisation vessel. A line wash of IPA (1.5 rel vols) was used to facilitate transfer.

Around 1% of the crystallisation solution was removed to provide a seed sample. This crystallised upon standing.

The crystallisation solution was cooled to 50° C., and then was cooled at 12° C./hour to 20° C. The seed was added when the crystallisation solution was at 40° C. The crystallisation solution was held at room temperature overnight.

The crystallised product was isolated by suction filtration. The resulting cake was washed with IPA (3.5 rel vols). The washed cake was then dried to constant mass in a vacuum oven at 50° C. to afford the sub-titled compound in 75% yield.

¹HNMR (400 MHz, DMSO-d⁶) 0.96 (3H, d, J=6), 1.02 (3H, d, J=6), 1.10 (1H, qd, J=12.5 and 4), 1.18 (1H, qd, J=12.5 and 4), 1.33 (1H, d, J=12.5), 1.60 (1H, m), 1.88 (1H, d, J=12.5), 2.49-2.66 (3H, m), 2.80 (1H, dd, J=15 and 5), 2.81 (3H, s), 2.91 (1H, m), 3.46 (1H, d, J=12), 3.57 (1H, d, J=12), 4.71 (1H, septet, J=6), 6.98 (2H, dd, J=8 and 1.5), 7.05 (1H, tt, J=9.5 and 1.5).

Method B: (using 2-(3,5-difluorophenyl)-5,5-dimethyl-1,3,2-dioxaborinane)

A catalyst solution was prepared by charging R-BINAP (0.035 mol eq) and bis(1,5-cyclooctadienerhodium chloride), (0.015 mol eq) to a vessel followed by THF (2.0 rel. vols). The mixture was stirred to achieve full dissolution.

To a larger reaction vessel was charged iso-propyl 3-(1-methanesulfonylpiperidin-4-yl)propenoate (1 mol eq), 2-(3,5-difluorophenyl)-5,5-dimethyl-1,3,2-dioxaborinane (1.5 mol eq) and potassium carbonate (0.2 mol eq). THF (10 rel vols) and IPA (1.1 mol eq) were then charged and the mixture was heated to 60° C. The catalyst solution was then added to this mixture, and the reaction mixture was held at 60-66° C. for 2 hours. The crude reaction mixture was concentrated in vacuo. The residue was largely dissolved into MTBE, and this solution was filtered through a pad of silica. The resulting solution was concentrated in vacuo and was triturated using iso-hexane and MTBE. The resulting solid was collected by filtration, and dried overnight in a vacuum oven at 40° C. The title compound was afforded in 67% yield.

### Step 6.

### Preparation of (3R)-3-(3,5-difluorophenyl)-3-[1-(methylsulfonyl)piperidin-4-yl]propan-1-ol

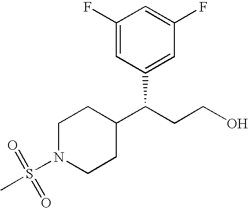

Diisobutylaluminium hydride (1M in tetrahydrofuran (DIBAL-H) (5.8 litres, 3.5 eq) was added dropwise over 45 minutes, to a solution of iso-propyl (3R)-3-(3,5-difluorophenyl)-3-[1-(methylsulfonyl)piperidin-4-yl]propanoate (646 g, 1.0 eq) in tetrahydrofuran (6.5 litres, 10 vol) at 0° C., keeping the temperature below 5° C. The reaction was stirred at 0° C. for 3 hours. The reaction was cooled to −15° C. Methanol (646 ml, 1 vol) was added dropwise to the reaction over 15 minutes. The mixture was stirred for 30 minutes until it had cooled back to −10° C.

An aqueous saturated solution of sodium potassium tartrate tetrahydrate (2900 g, 4.5 wt) in water (8.1 litres, 12.5 vol) was then added very carefully, keeping the temperature below 10° C. (exothermic −10° C.-+5° C., when the precipitate starts to form the exotherm increases dramatically).

Ethyl acetate (6.5 litres, 10 vol) was then added and the mixture stirred at room temperature for 30 minutes. This was then filtered through a pad of celite and washed through with ethyl acetate (6.5 litres, 10 vol). The aqueous layer was separated and extracted with ethyl acetate (2×10.0 litres). The organics were combined and washed with 50% water/brine (2×16.0 litres), dried (magnesium sulphate) and filtered. The volume was reduced in vacuo to half and then this was passed through a silica pad (˜1000 g, ˜1 wt) washing through with ethyl acetate (8.0 litres, 8 vol) and finally the solvent was removed in vacuo to give a white solid. Recrystallisation from ethyl acetate/isohexane gave the subtitled compound as a white solid (96%).

¹H NMR (400 MHz, DMSO) δ 0.96-1.23 (2H, m), 1.26-1.42 (1H, m), 1.51-1.78 (2H, m), 1.85-2.03 (2H, m), 2.42-2.72 (3H, m), 2.86 (3H, s), 2.99-3.14 (1H, m), 3.19 (1H, qd), 3.45 (1H, d), 3.57 (1H, d), 4.38 (1H, t), 6.84-7.13 (3H, m)

### Step 7.

### Preparation of (3R)-3-(3,5-difluorophenyl)-3-(1-methylsulfonyl-4-piperidyl)propanal

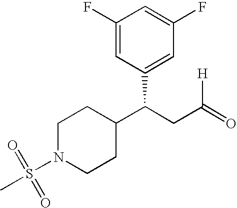

To a mixture of (3R)-3-(3,5-Difluorophenyl)-3-[1-(methylsulfonyl)piperidin-4-yl]propan-1-ol (258 g, 1.0 eq), sodium acetate (114 g, 1.8 eq) and tetra-methylpiperidine-N-oxide (1.2 g, 0.01 eq) in dichloromethane (5.0 litres, 20 vol), cooled to −5° C., was added a suspension of trichloroisocyannuric acid (189 g, 1.05 eq) in dichloromethane (2.5 litres, 10 vol) over 20 minutes in batches of ˜50 g (exotherm seen −5° C.-+5° C.). The reaction was stirred at 2° C. for 90 minutes. The reaction was filtered and washed through with dichloromethane (2.5 litres, 10 vol). The solvent was removed in vacuo to give a redish residue (308 g). The residue was dissolved in dichloromethane (500 ml) and a fine solid precipitate was filtered off through a pad of celite (250 g)/silica (250 g) (celite on the bottom) washing through with 30% ethyl acetate/dichloromethane (5.0 litres, 20 vol). The solvent was removed in vacuo to leave a yellow oil which was purified on a Novasep 1.5 kg silica column, eluting initially with 5% ethyl acetate/dichloromethane then a gradient up to 30% ethyl acetate/dichloromethane. The product fractions gave the subtitled compound as a white solid (174 g, 68% yield).

¹H NMR (400 MHz, DMSO) δ 0.99-1.24 (2H, m), 1.37 (1H, d), 1.60 (1H, m), 1.84 (1H, d), 2.44-2.68 (2H, m), 2.73-3.02 (5H, m), 3.06-3.17 (1H, m), 3.54 (2H, m), 6.94-7.13 (3H, m), 9.55 (1H, s).

### Intermediate 1.

### Preparation of 2,2-difluoropropanehydrazide

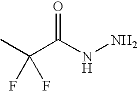

To a solution of hydrazine monohydrate (186 ml) in ethanol (2.5 L), cooled to −10° C., was added ethyl 2,2-difluoropropanote (500 g) over 45 minutes, keeping the temperature below 15° C. The reaction was then raised to 25° C. overnight and then warmed to 35° C. for 2 hours. The reaction was then reduced in vitro and azeotroped with toluene twice to give a solid. This was filtered and washed with diethyl ether/iso-hexane to give the titled product.

Yield (384 g, 85%).

¹H NMR (400 MHz, CDCl₃) δ 1.8 (3H, t), 3.8 (2H, bs), 7.9 (1H, bs)

### Intermediate 2.

### Preparation of 4-[3-(1,1-difluoroethyl)-5-methyl-1,2,4-triazol-4-yl]piperidine

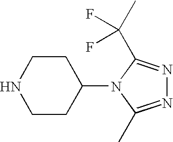

### Step 1.

### Preparation of N-(1-benzyl-4-piperidyl)acetamide

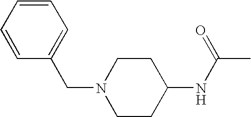

1-benzylpiperidin-4-amine (400 g) was suspended in dichloromethane (1.5 L) at room temperature and acetic anhydride (225.3 g) was added at such a rate to bring the reaction to a steady reflux. The reaction was then refluxed for 1 hour before cooling to 10° C. and adding 4M sodium hydroxide solution. The dichloromethane layer was the separated and dried over magnesium sulphate, filtered and reduced in vacuo until the product started to crystallise out of solution. Diethyl ether was then added to precipitate out the product, which was then stirred until cool then filtered to give a white solid. Yield 478 g, (98%).

¹H NMR (400 MHz, CDCl₃) δ 1.45 (2H, m), 1.9 (2H, m), 1.95 (3H, s), 2.15 (2H, m), 2.8 (2H, m), 3.45 (2H, s), 3.8 (1H, m), 5.4 (1H, bs), 7.3 (5H, m).

### Step 2.

### Preparation of 1-benzyl-4-[3-(1,1-difluoroethyl)-5-methyl-1,2,4-triazol-4-yl]piperidine

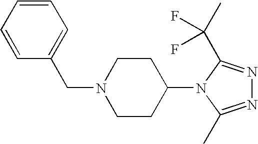

A solution of N-(1-benzyl-4-piperidyl)acetamide (478.0 g, 1.0 eq) in dichloromethane (7.2 litres, 15 vol) was added dropwise to a solution of phosphoruspentachloride (557 g, 1.3 eq) in dichloromethane (9.6 litres, 20 vol) at 0° C. over 20 minutes (a slight exotherm observed 0-5° C.). The reaction was left stirring at 0° C. for 30 minutes before being warmed to 25° C. and left to stir for 2 hours. The reaction was again cooled to 0° C. and a solution of 2,2-difluoropropanohydrazide (383 g, 1.5 eq) in dichloromethane (4.8 litres, 10 vol) was added dropwise over 30 mins, the reaction was warmed to 25° C. and stirred for 18 hours. The reaction was cooled to 0° C. and basified with 2M aqueous sodium hydroxide (11.5 litres, 24 vol) to pH12. The organic layer was separated and the aqueous layer extracted with dichloromethane (12.0 litres, 25 vol). All the organics were combined and dried (magnesium sulphate), filtered and the solvent removed in vacuo to give an off white solid: 675 g. The solid was slurried in toluene (20.0 litres, 40 vol), acetic acid (480 ml, 1 vol) was added and the reaction heated to reflux (˜105° C.) for 3 hours. The reaction was cooled to room temperature and stirred for 18 hours. The mixture was then reduced in volume to one quarter. This was basified with 2M aqueous sodium hydroxide (10.0 litres, 21 vol) and extracted with dichloromethane (2×7.5 litres, 2×16 vol). The organics were combined and washed with 50% brine/water (13.0 litres, 27 vol), dried (magnesium sulphate), filtered and the solvent removed in vacuo to give the subtitled compound as an off-white solid: 615 g, 93% yield.

¹H NMR (400 MHz, DMSO) δ 1.71-1.90 (2H, m), 1.95-2.29 (7H, m), 2.46-2.66 (3H, m), 2.97 (2H, d), 3.46-3.63 (2H, m), 4.31 (1H, q), 7.06-7.43 (5H, m).

### Step 3.

### Preparation of 4-[3-(1,1-difluoroethyl)-5-methyl-1,2,4-triazol-4-yl]piperidi

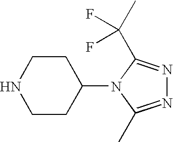

To a solution of 1-benzyl-4-[3-(1,1-difluoroethyl)-5-methyl-1,2,4-triazol-4-yl]piperidine (615 g) in ethanol (6.15 L) under an argon atmosphere was added 10% palladium on carbon (123 g). The resulting mixture was hydrogenated using pressure of 5Barr at 70° C. for 3 hours. The mixture was cooled and filtered through a pad of Celite, washing through with further amounts of ethanol. The organics were removed in vacuo and the resulting solid was azeotroped with toluene (2×1.2 L) to give the title compound (436 g, 99% yield).

¹H NMR (400 MHz, DMSO) δ 1.66-1.80 (2H, m), 1.91-2.06 (2H, m), 2.18 (3H, t), 2.43-2.62 (5H, m), 3.02-3.14 (2H, m), 4.38 (1H, q)

### EXAMPLE 2

The ability of the compound of the present invention to inhibit the binding of MIP-1β (CCL-4) was measured:

An allo-reactive T cell line was generated by exposure of human peripheral blood mononuclear cells (PBMCs) to L-DR4/B7 fibroblasts (immobilised with glutaraldehyde fixation and irradiation) and subsequent expansion with anti-CD3 and IL-2 for 14 days. The resultant Allo-T cells were frozen. When required, the cells were grown and re-challenged with irradiated HLA-DR4+ve PBMCs and expanded with anti-CD3 and IL-2. After 21 to 34 days culture, the membranes were prepared from the cells. These membranes were incubated in 96-well plates with 2 nM of the radio-labelled CCR5 antagonist [³H]1-{(3R)-3-(3,5-difluorophenyl)-3-[4-(methylsulfonyl)phenyl]propyl}-4-(2-{[4-(methylsulfonyl)phenyl]sulfonyl}ethyl)piperidine and various concentrations of the compound of the invention for 2 hours at room temperature. The plates were then harvested onto GF/B filter plates (which had been pre-soaked soaked in 0.3% PEI containing 0.2% BSA for 10 min at 4° C.) using a Packard Unifilter harvester using 10 wash steps. The amount of [³H]1-{(3R)-3-(3,5-difluorophenyl)-3-[4-(methylsulfonyl)phenyl]propyl}-4-(2-{[4-(methylsulfonyl)phenyl]sulfonyl}ethyl)piperidine retained on the filter plates was determined by scintillation counting. Competition curves were obtained for the compound of the invention and the concentration which displaced 50% of bound 1-{(3R)-3-(3,5-difluorophenyl)-3-[4-(methylsulfonyl)phenyl]propyl}-4-(2-{[4-(methylsulfonyl)phenyl]sulfonyl}ethyl)piperidine was calculated (IC₅₀).

Results from this test for 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine and Comparator Compound A of the invention are presented in Table I.

TABLE I

| Compound. | IC₅₀(μM) |
|---|---|
| Comparator Compound A | 0.039 |
| 4-[3-(1,1-difluoroethyl)-5-methyl-4H-1,2,4-triazol-4-yl]-1- | 0.0034 |
| {(1R,3R)-3-(3,5-difluorophenyl)-1-methyl-3-[1- | |
| (methylsulfonyl)piperidin-4-yl]propyl}piperidine | |

### EXAMPLE 3

The ability of the compound of the present invention to inhibit the chemotaxis of T-cells in response to MIP-1β (CCL-4) was measured:

An allo-reactive T cell line was generated by exposure of human peripheral blood mononuclear cells (PBMCs) to L-DR4/B7 fibroblasts (immobilised with glutaraldehyde fixation and irradiation) and subsequent expansion with IL-2 and anti-CD3. When required, the cells were grown and re-challenged with irradiated HLA-DR4+ve PBMCs and expanded with anti-CD3 and IL-2. The cells were used between day 21 and 34. 1 nM of MIP-1β plus varying concentrations of the CCR5 antagonist [³H]1-{(3R)-3-(3,5-difluorophenyl)-3-[4-(methylsulfonyl)phenyl]propyl}-4-(2-{[4-(methylsulfonyl)phenyl]sulfonyl}ethyl)piperidine were placed in assay buffer in the bottom of a 96-well Neuroprobe Chemo TX chemotaxis plate and cells that had been loaded with a fluorescent dye and pre-incubated with varying concentration of the CCR5 antagonist were pipetted onto the surface of a 5 micron pore size membrane according to the manufacturers specifications. After 1 hour of incubation with the compound of the invention at 37° C., the unmigrated cells were washed off the surface of the plate with PBS and the unmigrated cells quantitated by reading on a 96-well fluorescent plate reader. Inhibition curves were obtained for the compound of the invention and the concentration which inhibited 50% of the chemotactic response was calculated (IC₅₀).

Results from this test for 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine and Comparator Compound A of the invention are presented in Table II.

TABLE II

| Compound. | IC₅₀(μM) |
|---|---|
| Comparator Compound A | 0.068 |
| 4-[3-(1,1-difluoroethyl)-5-methyl-4H-1,2,4-triazol-4-yl]-1- | 0.0021 |
| {(1R,3R)-3-(3,5-difluorophenyl)-1-methyl-3-[1- | |
| (methylsulfonyl)piperidin-4-yl]propyl}piperidine | |

1. 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine (I): 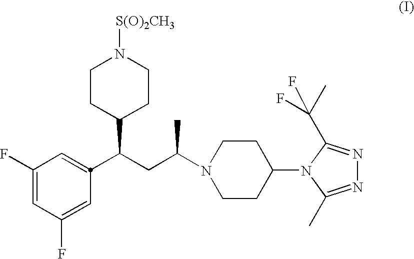
or a pharmaceutically acceptable salt thereof.

2. A process for the preparation of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine as claimed in claim 1, or a pharmaceutically acceptable salt thereof, which comprises:
reaction of a compound of formula (II): 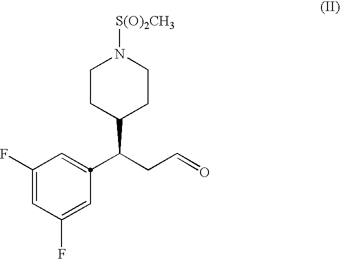
with a compound of formula (III) in the presence of an appropriate triazole: 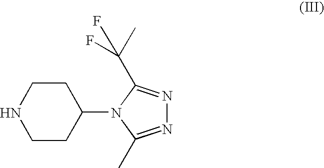
followed by reaction with an appropriate organometallic reagent.

3. A pharmaceutical composition which comprises 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine as claimed in claim 1, or a pharmaceutically acceptable salt thereof, and a pharmaceutically acceptable adjuvant, diluent or carrier.

4. 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine as claimed in claim 1, or a pharmaceutically acceptable salt thereof, for use as a medicament.

5. The use of 4-[3-(1,1-difluoroethyl)-5-methyl-4H-1,2,4-triazol-4-yl]-1-{(R,3R)-3-(3,5-difluorophenyl)-1-methyl-3-[1-(methylsulfonyl)piperidin-4-yl]propyl}piperidine as claimed in claim 1, or a pharmaceutically acceptable salt thereof, in the manufacture of a medicament for the treatment of a CCR5 mediated disease state.

6. A method of treating a CCR5 mediated disease state comprising administering to a patient in need of such treatment an effective amount of 4-[3-(1,1-difluoroethyl)-5-methyl-4H-1,2,4-triazol-4-yl]-1-{(1R,3R)-3-(3,5-difluorophenyl)-1-methyl-3-[1-(methylsulfonyl)piperidin-4-yl]propyl}piperidine as claimed in claim 1.
